(19) United States
(12) Patent Application Publication
(10) Pub. No.: US 2021/0341445 A1
(43) Pub. Date: Nov. 4, 2021

(54) METHOD FOR MONITORING CHANGE OF VEGETATION WATER CONSERVATION

(71) Applicant: Institute of Geochemistry, Chinese Academy of Sciences, Guiyang (CN)

(72) Inventors: Xiaoyong BAI, Guiyang (CN); Shijie WANG, Guiyang (CN); Luhua WU, Guiyang (CN); Fei CHEN, Guiyang (CN); Miao ZHOU, Guiyang (CN); Yichao TIAN, Guiyang (CN); Guangjie LUO, Guiyang (CN); Qin LI, Guiyang (CN); Jinfeng WANG, Guiyang (CN); Yuanhuan XIE, Guiyang (CN); Yujie YANG, Guiyang (CN); Chaojun LI, Guiyang (CN); Yuanhong DENG, Guiyang (CN); Zeyin HU, Guiyang (CN); Shiqi TIAN, Guiyang (CN); Qian LU, Guiyang (CN); Chen RAN, Guiyang (CN); Min LIU, Guiyang (CN)

(21) Appl. No.: 16/863,114

(22) Filed: Apr. 30, 2020

(51) Int. Cl. G01N 33/00

(52) U.S. Cl. CPC G01N 33/0098

(57) ABSTRACT

The present invention relates to a method for monitoring a change of vegetation water conservation. The method includes: obtaining global land water storage change data, precipitation, actual evapotranspiration, soil moisture storage, snowmelt, snow water storage, surface water storage, groundwater storage, change in surface and groundwater resources, litterfall interception water storage, average natural water content, maximum water holding capacity and litterfall accumulation; preprocessing the above data, and calculating a change of vegetation canopy water storage; calculating a change of litterfall interception water storage; calculating a change of soil moisture storage; and determining a water conservation change according to the change of vegetation canopy water storage, the change of litterfall interception water storage and the soil moisture change. The method provides new technical support and reference for the evaluation of ecological effects and water conservation during ecological restoration.

### TECHNICAL FIELD

The present invention relates to the field of ecological environment monitoring and surface and groundwater resource utilization, and in particular, to a method for monitoring a change of vegetation water conservation.

### BACKGROUND

The change of vegetation water conservation reflects the ecological benefits of vegetation restoration, and to some extent reflects the evolution of the ecosystem. At the present, the vegetation water conservation is calculated based on the sum of vegetation canopy water storage, litterfall interception water storage and soil moisture storage. The calculation models have the following limitations: First, the vegetation canopy water storage is mainly characterized by the maximum forest canopy interception water storage, which is inaccurate. In addition, the calculation model of the maximum vegetation canopy interception water storage is too simple. It only considers the average maximum water holding depth per leaf area, vegetation coverage and leaf area index. The maximum water holding depth per leaf area has a significant spatial difference due to different types of vegetation and regions, and is difficult to monitor. Due to the lack of monitoring data, this calculation method is only applicable for small regions. For large regions, the calculation cannot realize the spatialization of the average maximum water holding depth per leaf area, but can only adopt an average value, which has a large human interference and leads to a large error. Therefore, this calculation method is not conducive to the subsequent spatial-temporal dynamic evaluation of water conservation. Second, the calculation model of the change of soil moisture storage only considers soil depth and non-capillary porosity, and ignores the spatial difference of the soil thickness (which is often taken as 0.4 m). In addition, the non-capillary porosity is related to soil particle size, soil structure, soil gas exchange and crop growth. These factors have significant spatial differences and are difficult to monitor through model calculations. Therefore, the calculation model is not suitable for monitoring and research in large regions. In order to provide new technical support and reference for the evaluation of ecological effects and water conservation during ecological restoration, it is urgent to establish a new method for monitoring a change of vegetation water conservation.

### SUMMARY

An objective of the present invention is to provide a method for monitoring a change of vegetation water conservation. The present invention solves a technical defect that the existing techniques and models are difficult to monitor a spatiotemporal dynamic change of water conservation during vegetation restoration, making up for the blank of models.

To achieve the above purpose, the present invention provides the following technical solution.

A method for monitoring a change of vegetation water conservation includes:

obtaining global land water storage change data, as well as precipitation, actual evapotranspiration, soil moisture storage, snowmelt, snow water storage, surface water storage, groundwater storage, change in surface and groundwater resources, litterfall interception water storage, average natural water content, maximum water holding capacity and litterfall accumulation, where the global land water storage change data is obtained from Gravity Recovery and Climate Experiment (GRACE);

preprocessing the global land water storage change data, the precipitation, the actual evapotranspiration, the soil moisture storage, the snowmelt, the snow water storage, the surface water storage, the groundwater storage, the change in surface and groundwater resources, the litterfall interception water storage, the average natural water content, the maximum water holding capacity and the litterfall accumulation, to obtain preprocessed data;

calculating a change of vegetation canopy water storage by a general equation for global land water balance according to the preprocessed data;

calculating a change of litterfall interception water storage according to the preprocessed data;

calculating a soil moisture change according to the preprocessed data; and

determining a water conservation change according to the change of vegetation canopy water storage, the change of litterfall interception water storage and the soil moisture change.

Optionally, the change of vegetation canopy water storage is specifically calculated according to the preprocessed data by the following formula:

ΔCWS=ΔTWS−(ΔSnWS+ΔSWS+ΔSMS+ΔGWS)

=ΔTWS−(ΔSMS+ΔSnWS+ΔW/S)

=ΔTWS−ΔSMS−ΔSnWS−Δ[(Q_{SN}+P)−(ET+ΔSMS)]

where ΔW=Δ(Q_{SN}+P−ET−ΔSMS)×S=(ΔSWS+ΔGWS)×S,

ΔTWS=ΔSnWS+ΔCWS+ΔSWS+ΔSMS+ΔGWS; ΔCWS is a change of vegetation canopy water storage, mm; ΔSnWS is a change of snow water storage, mm; ΔSWS is a change of surface water storage, mm; ΔSMS is a change of soil moisture storage, mm; ΔGWS is a groundwater storage change, mm; ΔTWS is a change of total land water storage, mm; ΔW is a change in surface and groundwater resources, mm; P is a precipitation, mm; ET is an actual evapotranspiration, mm; the Q_{SN} is a snowmelt, mm; S is a pixel area, m².

Optionally, the change of litterfall interception water storage is specifically calculated according to preprocessed data by the following formula:

ΔCIS=Δ[(0.085Rₘ−0.1R₀)×M]

where, ΔCIS is a change of litterfall interception water storage, mm; R₀ is an average natural water content, g/kg; Rₘ is a maximum water holding capacity, g/kg; M is a litterfall accumulation, t/hm².

Optionally, the change of soil moisture storage is specifically calculated according to the preprocessed data by the following formula:

ΔSMS=SMSᵢ−SMSᵢ₋₁

where, SMSᵢ is soil moisture storage in an i^{th} month, mm; SMSᵢ₋₁ is soil moisture storage in an (i−1)^{th} month, mm.

Optionally, the water conservation change is specifically determined according to the change of vegetation canopy water storage, the change of litterfall interception water storage and the soil moisture change by the following formulas:

$$Δ {Q}_{WC} = Δ CWS + Δ CIS + Δ SMS = [ Δ TWS - Δ SMS - Δ SnWS - Δ [ ( {Q}_{SN} + P ) - ( ET + Δ SMS ) ] ] + Δ [ ( 0.085 {R}_{m} - 0.1 {R}_{0} ) \times M ] + Δ SMS$$

where: ΔQ_{WC} is a water conservation change, mm; ΔCWS is a change of vegetation canopy water storage, mm; ΔCIS is a change of litterfall interception water storage, mm; ΔSMS is a change of soil moisture storage, mm; R₀ is an average natural water content, g/kg; Rₘ is a maximum water holding capacity, g/kg; M is a litterfall accumulation, t/hm²; ΔTWS is a change of total land water storage, mm; ΔSnWS is a change of snow water storage, mm; Q_{SN} is a snowmelt, mm; P is a precipitation, mm; ET is an actual evapotranspiration, mm; S is a pixel area, m².

Optionally, the preprocessing specifically includes format conversion, image correction, cropping, registration, quality inspection and projection conversion.

According to specific embodiments provided by the present invention, the present invention discloses the following technical effects.

The present invention establishes a new method for monitoring a change of vegetation water conservation based on a water balance equation. First, the method estimates spatial occurrence characteristics of surface and groundwater resources on various pixel scales by considering the characteristics of precipitation, evapotranspiration, snow accumulation, snow coverage, soil moisture change, surface water and groundwater on each pixel scale. This method achieves the spatialization of surface and groundwater resources in a non-administrative region on a pixel scale. Second, this method abandons a traditional method/model which calculates vegetation canopy water storage by multiplying average maximum water holding depth per leaf area, vegetation coverage and leaf area index. Instead, this method calculates the vegetation canopy water storage by using snow water storage, surface water storage, soil moisture storage, groundwater storage and total land water storage by a general equation for global land water balance. This method avoids an error caused by the use of maximum canopy interception water storage to characterize the vegetation canopy water storage and also avoids artificial interference arising from the equalization of the average maximum water holding depth per leaf area. Therefore, this method improves the monitoring accuracy and realizes a spatial difference. Finally, this model solves a lack of monitoring data of average maximum water holding depth per leaf area on a large regional scale, and expands the scope of monitoring. Most importantly, the present invention uses high-efficiency and real-time remote sensing data, and on this basis, establishes a new method for monitoring a change of vegetation canopy water storage. This new method reduces the difficulty and time of monitoring, and provides support for the spatial-temporal dynamic evaluation of water conservation during vegetation restoration. In addition, the present invention abandons a traditional method for calculating soil moisture storage by multiplying a soil thickness (which can be taken as 0.4 m) by a non-capillary porosity. The present invention directly uses the remote sensing data of a soil water content as soil moisture storage, and provides a more accurate result and achieves a wider scope of monitoring and evaluation. By using remote sensing data, the present invention establishes a new method for monitoring a change of vegetation water conservation. This method provides new technical support and reference for the evaluation of ecological effects and water conservation during ecological restoration.

### BRIEF DESCRIPTION OF DRAWINGS

To describe the technical solutions in the embodiments of the present invention or in the prior art more clearly, the following briefly describes the accompanying drawings required for the embodiments. Apparently, the accompanying drawings in the following description show merely some embodiments of the present invention, and a person of ordinary skill in the art may still derive other accompanying drawings from these accompanying drawings without creative efforts.

FIG. 1 is a flowchart of a method for monitoring a change of vegetation water conservation according to the present invention.

### DETAILED DESCRIPTION

The following clearly and completely describes the technical solutions in the embodiments of the present invention with reference to accompanying drawings in the embodiments of the present invention. Apparently, the described embodiments are merely a part rather than all of the embodiments of the present invention. All other embodiments obtained by a person of ordinary skill in the art based on the embodiments of the present invention without creative efforts shall fall within the protection scope of the present invention.

An objective of the present invention is to provide a method for monitoring a change of vegetation water conservation. The present invention solves a technical defect that the existing techniques and models are difficult to monitor a spatiotemporal dynamic change of water conservation during vegetation restoration, making up for the blank of models.

To make the above objects, features, and advantages of the present invention more obvious and easy to understand, the present invention will be further described in detail with reference to the accompanying drawings and the detailed description.

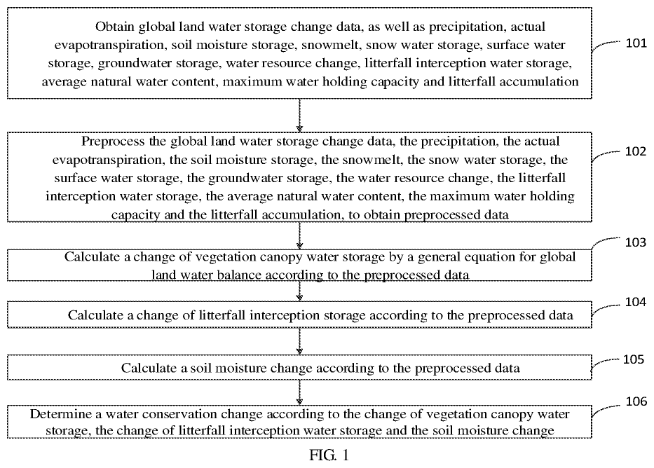

FIG. 1 is a flowchart of a method for monitoring a change of vegetation water conservation according to the present invention. As shown in FIG. 1, the method includes:

Step 101: obtain global land water storage change data, as well as precipitation, actual evapotranspiration, soil moisture storage, snowmelt, snow water storage, surface water storage, groundwater storage, change in surface and groundwater resources, litterfall interception water storage, average natural water content, maximum water holding capacity and litterfall accumulation.

In the present invention, the meteorological data is used to calculate a surface and groundwater resource storage, including the following monthly data: precipitation, actual evapotranspiration, snow water storage, and soil moisture content at a thicknesses of 0-10 cm, 10-40 cm, 40-100 cm and 100-200 cm, which are merged into annual data. These data are derived from a dataset of the Famine Early Warning Systems Network Land Data Assimilation System (FLDAS) (FLDAS Noah Land Surface Model L4 Global Monthly 0.1×0.1 degree (MERRA-2 and CHIRPS) V001 (FLDAS_NOAH01_C_GL_M) at GES DISC (https://ldas.gsfc. nasa.gov/FLDAS/)) available on the National Aeronautics and Space Administration (NASA) (https://www.nasa.gov/). They have a spatial resolution of 0.1°×0.1°, a monthly time resolution and a global spatial coverage (60S, 180W, 90N and 180E). In addition, global soil depth data is used to calculate a soil water content, which is derived from https://daac.ornl.gov/(spatial resolution 0.1°×0.1°) and https://www.isric.org/explore/soilgrids (250 m×250 m, 1 km×1 km, 5 km×5 km, and 10 km×10 km). The latest administrative division vector data in 2015 is derived from the Resource and Environment Data Cloud Platform of the Chinese Academy of Sciences (http://www.resdc.cn/) and the National Bureau of Surveying, Mapping and Geographic Information (http://www.sbsm.gov.cn/article/zxbs/dtfw/). The global land water storage change data is derived from GRACE Tellus website (https://grace.jpl.nasa.gov/data/get-data/). Global land snowmelt data is derived from the Global Land Data Assimilation System (GLDAS) at the Goddard Earth Sciences Data and Information Services Center (GES DISC) (GLDAS Noah Land Surface Model L4 Monthly 0.25×0.25 degree) (https://mirador.gsfc.nasa.gov/).

Step 102: preprocess the global land water storage change data, the precipitation, the actual evapotranspiration, the soil moisture storage, the snowmelt, the snow water storage, the surface water storage, the groundwater storage, the change in surface and groundwater resources, the litterfall interception water storage, the average natural water content, the maximum water holding capacity and the litterfall accumulation, to obtain preprocessed data.

The present invention utilizes a data assimilation method to convert a grid cell size of all raster data to the same spatial resolution based on Albers Equal-area Conic Projection (Krasovsky-1940-Albers). The present invention processes the global scale raster data by format conversion, image correction, cropping and quality inspection to finally obtain a climate element dataset of a study area. Based on these data set, the present invention first obtains a spatialized water resource occurrence characteristic according to a water balance equation, then calculates a change of vegetation canopy water storage, and finally calculates a change of vegetation water conservation.

Step 103: calculate a change of vegetation canopy water storage by a general equation for global land water balance according to the preprocessed data.

The water balance equation is:

S(Q_{SN}+P)=S(ET+ΔSMS)+R+G

where Q_{SN} is a snowmelt, mm; P is a precipitation, mm; ET is an actual evapotranspiration, mm; R is a runoff, m³; G is a groundwater recharge, m³; ΔSMS is a soil moisture change, mm; S is a pixel area, m.

The vegetation canopy water storage is calculated by the following formula:

[symbol]

ΔCWS=ΔTWS−(ΔSnWS+ΔSWS+ΔSMS+ΔGWS)

=ΔTWS−(ΔSMS+ΔSnWS+ΔW/S)

=ΔTWS−ΔSMS−ΔSnWS−Δ[(Q_{SN}+P)−(ET+ΔSMS)]

where ΔW=Δ(Q_{SN}+P−ET−ΔSMS)×S=(ΔSWS+ΔGWS)×S,

ΔTWS=ΔSnWS+ΔCWS+ΔSWS+ΔSMS+ΔGWS; ΔCWS is a change of vegetation canopy water storage, mm; ΔSnWS is a change of snow water storage, mm; ΔSWS is a change of surface water storage, mm; ΔSMS is a change of soil moisture storage, mm; ΔGWS is a groundwater storage change, mm; ΔTWS is a change of total land water storage, mm; ΔW is a change in surface and groundwater resources, mm; P is a precipitation, mm; ET is an actual evapotranspiration, mm; the Q_{SN} is a snowmelt, mm; S is a pixel area, m².

The snow water storage change SnWS is derived from a dataset of the FLDAS (FLDAS Noah Land Surface Model L4 Global Monthly 0.1×0.1 degree (MERRA-2 and CHIRPS) V001 (FLDAS_NOAH01_C_GL_M) at GES DISC (https://ldas.gsfc.nasa.gov/FLDAS/)) available on the NASA (https://www.nasa.gov/). It has a spatial resolution of 0.1°×0.1°, a monthly time resolution and a global spatial coverage (60S, 180W, 90N and 180E). Global land snowmelt data is derived from the GLDAS at the GES DISC (GLDAS Noah Land Surface Model L4 Monthly 0.25×0.25 degree) (https://mirador.gsfc.nasa.gov/).

The soil moisture change SMS uses soil water content raster data (soil depth 2 m) with a spatial resolution of 0.1°×0.1° from a dataset of the FLDAS (FLDAS Noah Land Surface Model L4 Global Monthly 0.1×0.1 degree (MERRA-2 and CHIRPS) V001 (FLDAS_NOAH01_C_GL_M) at GES DISC (https://ldas.gsfc.nasa.gov/FLDAS/)) available on the NASA (https://www.nasa.gov/).

Total surface and groundwater resources are calculated by the following formula:

W=R+G=S(Q_{SN}+P−ET−ΔSMS)

where, W is total surface and groundwater resources, m³; P is a precipitation, mm; ET is an actual evapotranspiration, mm; R is a runoff, m³; G is a groundwater recharge, m³; ΔSMS is a soil moisture change, mm; Q_{SN} is a snowmelt, mm; S is a pixel area, m².

The total surface and groundwater resources are calculated by the following formula:

ΔTWS=ΔSnWS+ΔCWS+ΔSWS+ΔSMS+ΔGWS

where, ΔCWS is vegetation canopy water storage, mm; ΔSnWS is snow water storage, mm; ΔSWS is surface water storage, mm; ΔSMS is soil moisture storage, mm; ΔGWS is groundwater storage, mm; ΔTWS is total land water storage, mm.

The total surface and groundwater resources are calculated by the following formula:

ΔW=Δ(Q_{SN}+P−ET−ΔSMS)×S=(ΔSWS+ΔGWS)×S

where ΔW is a change in surface and groundwater resources, m³; ΔSWS is surface water storage, mm; ΔGWS is groundwater storage, mm; P is a precipitation, mm; ET is an actual evapotranspiration, mm; ΔSMS is a soil moisture change, mm; Q_{SN} is a snowmelt, mm; S is a pixel area, m².

Step 104: calculate a change of litterfall interception water storage according to the preprocessed data.

Specifically, the calculation formula is as follows:

ΔCIS=Δ[(0.085Rₘ−0.1R₀)×M]

where, ΔCIS is a change of litterfall interception water storage, mm; R₀ is an average natural water content, g/kg; Rₘ is a maximum water holding capacity, g/kg; M is a litterfall accumulation, t/hm².

Step 105: calculate a soil moisture change according to the preprocessed data.

Specifically, the calculation formula is as follows:

ΔSMS=SMSᵢ−SMSᵢ₋₁

where, SMSᵢ is soil moisture storage in an i^{th} month, mm; SMSᵢ₋₁ is soil moisture storage in an (i−1)^{th} month, mm.

Step 106, determine a water conservation change according to the change of vegetation canopy water storage, the change of litterfall interception water storage and the soil moisture change.

Specifically, the calculation formula is as follows:

$$Δ {Q}_{WC} = Δ CWS + Δ CIS + Δ SMS = [ Δ TWS - Δ SMS - Δ SnWS - Δ [ ( {Q}_{SN} + P ) - ( ET + Δ SMS ) ] ] + Δ [ ( 0.085 {R}_{m} - 0.1 {R}_{0} ) \times M ] + Δ SMS$$

where: ΔQ_{WC} is a water conservation change, mm; ΔCWS is a change of vegetation canopy water storage, mm; ΔCIS is a change of litterfall interception water storage, mm; ΔSMS is a change of soil moisture storage, mm; R₀ is an average natural water content, g/kg; Rₘ is a maximum water holding capacity, g/kg; M is a litterfall accumulation, t/hm²; ΔTWS is a change of total land water storage, mm; ΔSnWS is a change of snow water storage, mm; Q_{SN} is a snowmelt, mm; P is a precipitation, mm; ET is an actual evapotranspiration, mm; S is a pixel area, m².

Each embodiment of the present specification is described in a progressive manner, each embodiment focuses on the difference from other embodiments, and the same and similar parts between the embodiments may refer to each other.

In this paper, several examples are used for illustration of the principles and implementations of the present invention. The description of the foregoing embodiments is used to help illustrate the method of the present invention and the core principles thereof. In addition, those skilled in the art can make various modifications in terms of specific implementations and scope of application in accordance with the teachings of the present invention. In conclusion, the content of the present specification should not be construed as a limitation to the present invention.

What is claimed is:

1. A method for monitoring a change of vegetation water conservation, wherein the monitoring method comprises:
obtaining global land water storage change data, as well as precipitation, actual evapotranspiration, soil moisture storage, snowmelt, snow water storage, surface water storage, groundwater storage, change in surface and groundwater resources, litterfall interception water storage, average natural water content, maximum water holding capacity and litterfall accumulation, wherein the global land water storage change data is obtained from Gravity Recovery and Climate Experiment (GRACE);
preprocessing the global land water storage change data, the precipitation, the actual evapotranspiration, the soil moisture storage, the snowmelt, the snow water storage, the surface water storage, the groundwater storage, the change in surface and groundwater resources, the litterfall interception water storage, the average natural water content, the maximum water holding capacity and the litterfall accumulation, to obtain preprocessed data;
calculating a change of vegetation canopy water storage by a general equation for global land water balance according to the preprocessed data;
calculating a change of litterfall interception water storage according to the preprocessed data;
calculating a soil moisture change according to the preprocessed data; and
determining a water conservation change according to the change of vegetation canopy water storage, the change of litterfall interception water storage and the soil moisture change.

2. The method for monitoring a change of vegetation water conservation according to claim 1,
wherein the change of vegetation canopy water storage is specifically calculated according to the preprocessed data by the following formula:
$$Δ CWS = Δ TWS - ( Δ SnWS + Δ SWS + Δ SMS + Δ GWS ) = Δ TWS - ( Δ SMS + Δ SnWS + Δ W \text{/} S ) = Δ TWS - Δ SMS - Δ SnWS - Δ [ ( {Q}_{SN} + P ) - ( ET + Δ SMS ) ]$$
$$wherein , Δ W = Δ ( {Q}_{SN} + P - ET - Δ SMS ) \times S = ( Δ SWS + Δ GWS ) \times S ,$$
ΔTWS=ΔSnWS+ΔCWS+ΔSWS+ΔSMS+ΔGWS; ΔCWS is a change of vegetation canopy water storage, mm; ΔSnWS is a change of snow water storage, mm; ΔSWS is a change of surface water storage, mm; ΔSMS is a change of soil moisture storage, mm; ΔGWS is a groundwater storage change, mm; ΔTWS is a change of total land water storage, mm; ΔW is a change in surface and groundwater resources, mm; P is a precipitation, mm; ET is an actual evapotranspiration, mm; the Q_{SN} is a snowmelt, mm; S is a pixel area, m².

3. The method for monitoring a change of vegetation water conservation according to claim 1, wherein the change of litterfall interception water storage is specifically calculated according to preprocessed data by the following formula:
ΔCIS=Δ[(0.085Rₘ−0.1R₀)×M]
wherein, ΔCIS is a change of litterfall interception water storage, mm; R₀ is an average natural water content, g/kg; Rₘ is a maximum water holding capacity, g/kg; M is a litterfall accumulation, t/hm².

4. The method for monitoring a change of vegetation water conservation according to claim 1, wherein the change of soil moisture storage is specifically calculated according to the preprocessed data by the following formula:
ΔSMS=SMSᵢ−SMSᵢ₋₁
wherein, SMSᵢ is soil moisture storage in an i^{th} month, mm; SMSᵢ₋₁ is soil moisture storage in an (i−1)^{th} month, mm.

5. The method for monitoring a change of vegetation water conservation according to claim 1, wherein the water conservation change is specifically determined according to the change of vegetation canopy water storage, the change of litterfall interception water storage and the soil moisture change by the following formulas:
$$Δ {Q}_{WC} = Δ CWS + Δ CIS + Δ SMS = [ Δ TWS - Δ SMS - Δ SnWS - Δ [ ( {Q}_{SN} + P ) - ( ET + Δ SMS ) ] ] + Δ [ ( 0.085 {R}_{m} - 0.1 {R}_{0} ) \times M ] + Δ SMS$$
wherein: ΔQ_{WC} is a water conservation change, mm; ΔCWS is a change of vegetation canopy water storage, mm; ΔCIS is a change of litterfall interception water storage, mm; ΔSMS is a change of soil moisture storage, mm; R₀ is an average natural water content, g/kg; Rₘ is a maximum water holding capacity, g/kg; M is a litterfall accumulation, t/hm²; ΔTWS is a change of total land water storage, mm; ΔSnWS is a change of snow water storage, mm; Q_{SN} is a snowmelt, mm; P is a precipitation, mm; ET is an actual evapotranspiration, mm; S is a pixel area, m².

6. The method for monitoring a change of vegetation water conservation according to claim 1, wherein the preprocessing specifically comprises format conversion, image correction, cropping, registration, quality inspection and projection conversion.
